(19) United States
(12) Patent Application Publication
(10) Pub. No.: US 2004/0147906 A1
(43) Pub. Date: Jul. 29, 2004

(54) Implantable interface system

(76) Inventors: Sophocles S. Voyiazis, Dallas, TX (US); Paul Kolostroubis, McKinney, TX (US); Laura Noelia Armas-Kolostrobis, McKinney, TX (US)

Correspondence Address: FISH & RICHARDSON P.C., 5000 BANK ONE CENTER, 1717 MAIN STREET, DALLAS, TX, 75201, US

(21) Appl. No.: 10/755,417

(22) Filed: Jan. 12, 2004

Related U.S. Application Data
(60) Provisional application No. 60/319,860, filed on Jan. 12, 2003.

(51) Int. Cl. A61K 9/22

(52) U.S. Cl. 604/891.1; 600/573; 600/562

(57) ABSTRACT

An interface between the external and internal environments associated with an essentially closed system, such as a human body, comprises an oral implant. The oral implant includes one or more chambers capable of containing materials delivered and/or receiving materials extracted by the implant. Micro- and nano-mechanical and electro-mechanical components, such as microfluidic pumps, perform the mechanics of the delivery and/or extraction of material via the oral implant. Miniature testing devices, such as a “lab-on-a-chip”, can be associated with the oral implant to perform in situ testing on material extracted via the implant. Communications means, including wireless communications, associated with the oral implant allow for remote control of the implant and/or remote reporting of information associated with the implant, such as test results.

### CROSS-REFERENCE TO RELATED APPLICATIONS

This application is related to co-pending provisional application serial No. 60/319,860, filed Jan. 12, 2003, which is incorporated herein by reference for any and all purpose.

### TECHNICAL FIELD

This invention relates to environment interfaces, and more particularly to an implantable interface system capable of providing an interface between the internal and external environments associated with a human body.

### BACKGROUND

There are many reasons that it would be advantageous to have a simple and effective interface between the internal and external environments associated with an essentially closed system, such as a human body. One clear advantage is associated with the efficacious transfer of materials and information between the external and internal environments associated with the body. Examples of such transfers include, without limitation, the introduction of materials (e.g., drugs) to the interior of the body, and the extraction of material (e.g., blood, tissue) from the interior of the body. More specifically, the taking of blood, tissue or other materials from a human for any reason (e.g., diagnostic testing), as well as the delivery of one or more drugs to a human body for treatment or prevention of various diseases and/or maladies, are common examples of such desired transfers. Numerous other examples exist, but these basic input and output functions associated with the introduction and extraction of materials between the external and internal environments of a human body will be used for purposes of example in this application. It is noted that while the examples provided in this application will include the use of a body, and, more specifically, a human body, the advantages and unique characteristics associated with present invention are applicable to, and therefore contemplated by, the present invention, any closed system, such as animals, plants and the like.

Traditional forms of drug administration have been problematic for physicians and patients alike. For example, non-compliant patients frustrate the proposed treatment of a particular ailment when instructions on taking medication(s), however complex and inconvenient, are not closely followed. Indeed, it is estimated that up to fifty percent of prescribed medications are not administered. Non-compliant patients not only potentially compromise the proposed treatment, but also leave the prescribing physician and/or researcher guessing about the effectiveness of the prescribed medicine. At the same time, many patients find it difficult at best to follow instructions for taking multiple medications and avoiding undesirable side effects. These common problems are magnified as patients increasingly receive medical care from multiple providers, all of which may have the patient on one or more medications with associated instructions and potential side effects.

Another disadvantage associated with traditional forms of drug administration is that relatively large doses of medication are needed to treat a particular ailment. Such large dosages are needed since a relatively large portion of the drug(s) administered is wasted as it/they travel through the digestive system of a patient. Only a minute portion of the total amount administered is eventually delivered to a targeted area of the body to fight a particular ailment. The requirement that such large dosages be used contributes to the escalating costs associated with medical care. Other forms of delivery have associated disadvantages. For example, drugs that are delivered intravenously (IV), especially the ones used for treatment of chronic diseases, create separate problems (e.g., infections) for patients who repeatedly undergo the unpleasant procedure of establishing an IV line. In extreme cases, patients have to endure a surgical procedure so that a subcutaneous port is created for drug administration.

Yet another problem associated with traditional forms of delivering medications to patients is the complete lack of specificity with respect to individual patients. Dosages for medications delivered via traditional methods and systems are determined for large groups of people (i.e., based upon renal function, liver function, age, race, weight, gender, etc.), and cannot account for the individual needs of a particular patient. As a result, the dosage that is most effective for vast majority of members of these large groups may be ineffective, or even detrimental, for any individual within the group.

Recent advances in drug delivery systems provide a relatively targeted approach as compared to the above-identified prior art systems and methods. One example of such recent advances is ocular drug delivery systems. However, even with these advanced systems and methods there remain problems. For example, ocular drug implants are drug specific (i.e., do not lend themselves for use with more than one type of medication (e.g., gancyclovir)), require invasive procedures for placement, and preclude self-maintenance (e.g., refilling of medication).

On the other side of the equation, the extraction of materials from a body provides another area for advantageous application of the present invention system. Common examples of the need for simple extraction of materials from a body include the testing of blood and tissue from within the body, and the monitoring of vital signs (e.g., blood pressure). Traditionally, such extractions represent a time consuming, inefficient and potentially painful experience. For extraction of actual materials, relatively large volumes of such materials are required by traditional systems and methods. Additionally, such prior art systems and methods require disruption of normal activities to extract the materials, providing only a “snap-shot” in time of materials extracted/vital signs monitored. Finally, intrusive, if not invasive, actions are needed for the collection of such materials. The dynamic nature of the present invention system is therefore ideally suited for patients requiring extraction of materials and/or continuous monitoring of vital signs information.

Recent advances in testing systems have resulted in self-contained testing units, known colloquially as “lab-on-a-chip” units. These units can perform analysis and reporting of various system fluids and functions of a body and even report such information to remote locations. The incorporation of such advances into the present invention system is suitable, and therefore contemplated.

There remains a need for an interface system capable of providing a simple and effective interface between the external and internal environments associated with a closed system, such as a human body, said system being capable of introducing and extracting material and information via said interface for a variety of purposes and uses and subject to remote control and capable of providing information to remote locations.

### SUMMARY

The present invention provides an interface between the external and internal environments associated with a substantially closed system, such as a human body. In a preferred embodiment, the present invention system is an implantable device, preferably in the form of a dental implant.

The present invention system comprises an oral implant including at least one substantially hollow cavity. The oral implant is implanted in one of several potential target sites within the patient to act as an interface between the external and internal environments of the patient. Implantation of the device occurs in one of several methods known in the art. Any suitable method of locating and implanting the present invention system so that it can function as an interface between the interior of the patient and the external environment is contemplated by this invention. In this embodiment, translocation (e.g., absorption, diffusion or other transmission) of material between the internal and external environments associated with the user occurs via the highly vascularized tissue surrounding the implant area.

Once in place, the system acts as an interface or portal through which materials can be introduced into the patient, on the one hand, and through which materials can be extracted from the patient, on the other hand. Embodiments of the present invention system contemplate both single purpose implants (i.e., only designed to introduce materials or to extract materials) and multipurpose implants (i.e., designed to both introduce and extract materials).

Common examples of material introduction include delivery of drugs, medications, immunizations, supplements and the like. Common examples of material extraction via the implant include testing of blood, bodily fluids, tissue and the like.

The one or more chambers of the implant receive and contain one or more materials (e.g., medications) to be delivered to the patient via the implant. Upon the occurrence of a predetermined set of circumstances, including manual manipulation, the materials contained within the chamber(s) can be selectively provided to the patient via the implant. Optionally, certain materials can be delivered over time via degradation (e.g., use of a biodegradable polymer).

Conversely, the one or more chambers of the implant can include means for extracting blood or the like via the implant, such as a microfluidic device. Such extracted material can either be manipulated or otherwise acted upon (e.g., performance of one or more tests) in situ, or the material can merely be held until it is removed from the chamber for further storage and/or processing. This embodiment of the implant system performs as a “laboratory-in-the-patient” device. As such, the functionality of the system described above in connection with the delivery of material is “reversed”, as far as the fluid flow is concerned. As outlined in further detail herein, minute pumps, springs, and similar mechanical or electro-mechanical components can be employed to assist in the delivery and/or extraction and further treatment or processing of material via the implant. Advances in nanotechnology, including the development of micromechanical machines (MEMS) and nanomechanical machines (NEMS) are particularly suited for use with the present invention system.

Preferentially, access to the one or more chambers within the implant is such that the individual associated with the implant could, if desired, provide simple maintenance (e.g., change or “refill” medications) for the implant. Such a feature would have particular relevance to embodiments utilized by those with chronic illnesses (e.g., diabetes). In various embodiments, access to the one or more chambers of the implant could be accomplished via hinged covers, removable covers, threaded covers, sliding covers and the like.

For various embodiments of the present invention, it may be advantageous for the implant to include means for communicating with one or more remote locations (a communications interface). For example, in an implant including a microfluidic device capable of extracting and testing blood or other bodily fluids, once a “sample” is extracted and tested, the results of the test may be transmitted to a remote location, like a health care provider. The use of known wireless communications systems provides this unique function of the implant. In a separate, but related, embodiment, the results of such testing of blood may be used to provide automatic feedback to the implant device to cause adjustments in the delivery of material(s) via the implant. Use of such a device is ideal for diabetes patients, for example, as periodic monitoring of blood sugar could provide the feedback for the delivery of insulin.

Importantly, the only invasive procedure associated with use of an implant of this design is the initial implanting of the device, a minimally invasive procedure that rarely lasts more than a half an hour. Utilizing the removable and replaceable capsule/chamber of the present invention system, the maintenance of the implant system is extremely easy and painless to the individual with whom the implant is associated. Exemplary maintenance functions associated with the implant include the replacement and refilling of medications to be delivered, the removal and cleaning of materials to be tested and/or already tested, replacement of reagents used in testing, maintenance of any electrical and electro-mechanical components, and the like.

Playing a central role in embodiments of the present invention system is the micro- or nano-pump that either extracts or delivers material via the implant. It is noted that any device capable of moving fluids or materials from one point to another, via any means, including, without limitation, the screening out of undesired forces present in the environment (as motion is often accomplished in the context of nano-environments), and that otherwise conforms to the size constraints, biocompatibility and performance requirements, may be used with the present invention system. It is noted that even relatively passive fluid flow, for example that resulting from degradation of an encapsulated polymer via enzymatic action, is suitable for use with the present invention implant system.

It is important to note that this system can accommodate variable dosage profiles. Additionally, multiple drugs can be delivered if the one or more chambers are subdivided and fitted with the appropriate delivery systems, as described in further detail below. Optionally, the delivery of these drugs can be programmed and/or controlled from a remote source, such as a health care provider or the like. This feature of the present invention renders the system a perfect candidate for the administration of hormones, antibiotics, vaccines, advanced life support agents (e.g., epinephrine, dopamine, etc), enzymes chemotherapeutic agents, and even low dose radioactive devices.

One advantageous use of the present invention system is as an insulin pump. The implant system provides the human associated with the implant insulin in either an “unintelligent” manner (via simple provision of a predetermined quantity of insulin) or, preferably, in an “intelligent” manner via the periodic (or even constant) monitoring of blood sugar via the interface system, and, utilizing the results of same, the provision of specific amounts of insulin based upon such monitored results. Although insulin is described herein, the present invention system can be used to deliver any one or more medications or other therapeutic materials, if desired.

Apart from the specific potential implementations identified above, the interface system of the present invention can also be used as an automated immunization/treatment system, the use of which could protect soldiers, even at remote and diverse locations, against chemical or biochemical warfare. Such a system would include a reservoir for containing one or more materials, such as drugs, to be administered to the person with whom the system was associated under certain conditions. For example, as soldiers are deployed over a large geographic area during combat or the like, general command could determine that the troops were subject to an imminent chemical or biological attack. Utilizing any number of communications systems, including, wireless systems, instructions for the delivery of one or more specific antidotes and/or medications could be sent out by command and received by the present invention system. The result would be the immediate delivery of the appropriate type and level of medication(s)/antidote(s) to far-flung individuals, even those who were unaware (e.g., asleep, unconscious, etc.) of any potential attack. Such a system would allow for appropriate and immediate protection/treatment of such individuals without the need for disruption of those individuals' current responsibilities and/or movement of such individuals or travel for medical personnel to accomplish this result. Although the example of a combat embodiment has been described above, an equally important use of such an embodiment of the present system is in the context of protection from terrorism. Any individual could employ such a system to provide a quick and appropriate administration of antidote(s)/treatment(s) depending upon the terrorism threat faced/experienced by that individual. Parents, guardians and/or caregivers could control (even remotely) such systems for their children, invalid or incapacitated charges.

Another exemplary use of the present invention interface system is as a tracking device for humans (e.g., the elderly, children, Alzheimer patients, etc.), in which miniaturized electronics could be employed to provide transmission of information, potentially coordinated with global tracking systems, such as global positioning systems, about the human associated with the interface. Current tracking devices for use in these contexts suffer from a variety of disadvantages, including relatively large and bulky dimensions, and a lack of tamper resistance.

Yet another exemplary use of the present invention interface system is as a “black-box” capable of monitoring one or more parameters about the human associated therewith, and reporting same, either in real time, or as a recorded history over time. Such a device could be used, for example, to monitor incarcerated individuals or even provide the monitoring necessary to allow qualifying individuals to be under “house arrest” versus actual incarceration. Other uses of such a system, for example to monitor the vital signs of astronauts, are contemplated by the present invention system.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a plan view of one embodiment of the implant system of the present invention configured as a drug delivery system;

FIG. 2 is a plan view of another embodiment of the implant system of the present invention as a drug delivery device including a biodegradable plug;

FIG. 3 is a partial cross section of another embodiment of the implant system of the present invention as a drug delivery system including additional drug storage;

FIG. 4 is a plan view of one embodiment of the implant system of the present invention configured as a blood test system;

FIG. 5 is a partial cross section of another embodiment of the implant system of the present invention as a blood test system including a lab-on-a chip;

FIG. 6 is a partial cross section of one embodiment of the implant system of the present invention including a drug delivery system and a fluid test system;

FIG. 7 is a cross section of a micropump including a magnetically-actuated membrane;

FIG. 8A is a cross section of the membrane portion of the micropump of FIG. 7, shown in an original, resting state;

FIG. 8B is a cross section of the membrane portion of the micropump of FIG. 7, shown in an actuated state;

FIG. 9 is a diagram of the general electronics of an embodiment of the oral implant of the present invention; and

FIG. 10 is a flowchart illustrating the logic flow for an embodiment of the oral implant of the present invention.

Like reference symbols in the various drawings indicate like elements.

### DETAILED DESCRIPTION

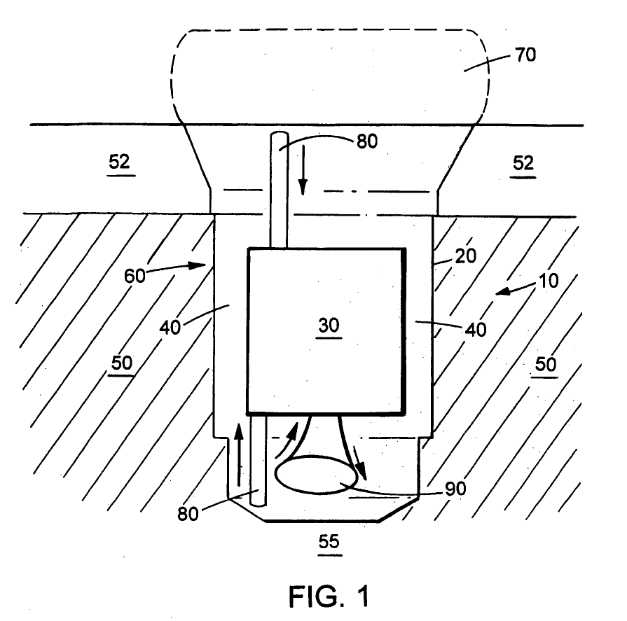

Referring now to FIG. 1, there is shown an implantable interface in the form of an oral implant 10 in accordance with one embodiment of the present invention. The oral implant 10 comprises a main body 20, a pump 30 and a chamber 40.

The main body 20 of the oral implant 10 includes two main components: a lower component 60 that serves as an anchor within maxillary or mandibular bone 50 of a patient (below the gums 52) and stays in place throughout the life of the oral implant 10; and an upper component 70 that is removably attached to the lower component 60 so that maintenance (e.g., refilling of medication(s), repair of internal components, etc.) can be performed on the internal components of the oral implant 10. The upper component 70 could be a crown replacing a missing tooth, or merely a container of any suitable shape and size. The upper component 70 is removably attached to the lower component 60 via any suitable means, including threaded screw assembly, snaps, hooks, or the like. Any suitable means for removably joining the lower component 60 and the upper component 70 may be used with the present invention oral implant 10. For purposes of this application, the term “maintenance” is used to refer to any activity associated with accessing and/or manipulating the oral implant 10, such as removing, replacing or refilling medication, repairing or maintaining any internal component or structure of the oral implant, accessing materials extracted, gathering data or other information from the oral implant 10, and the like.

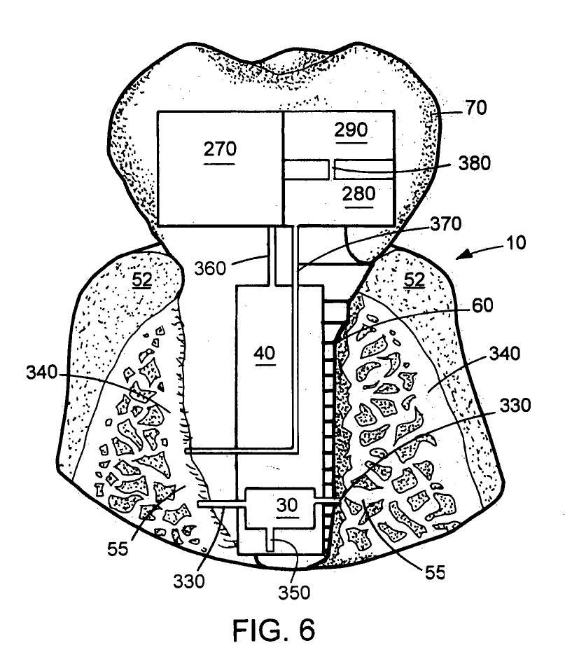

Associated with the pump 30 are draw microchannels 80 and an output microchannel 90. The draw microchannels 80 function as small conduits through which the contents of the chamber 40, such as medication, are drawn via suction from the chamber 40. The suction is provided by the pump 30. The contents of the first chamber 40 are drawn via draw microchannels 80 by the pump 30 and provided to the capillary bed, or a transmission site 55 associated with the lower component 60 (See FIG. 6) via the output microchannel 90. After release from the oral implant 10 via the output microchannel 90, absorption distributes the released medication to the adjacent tissue and, via circulation, it is distributed to the rest of the body.

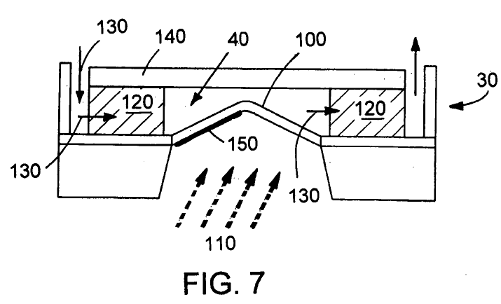

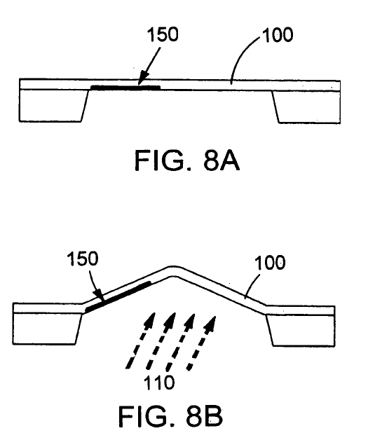

As illustrated in FIGS. 7, 8A and 8B, the pump 30 is preferably a microfluidics pump or the like including a membrane 100 that is actuated in response to a magnetic field 110, but can be any pump available that conforms to the size constraints, biocompatibility and performance requirements of the specific desired application.

Now referring to FIG. 8A, the membrane 100 is shown in a resting (i.e., non-actuated) state. When actuated by the magnetic field 110, the membrane 100 deflects (FIG. 8B) and pushes fluid out of the pump chamber 40 (FIG. 7). Uni-directional diffusers 120 placed at the inlet and outlet of the pump chamber 40 then control the direction of fluid flow, illustrated in FIG. 7 with arrows 130. The uni-directional diffusers are preferably polymer based, but can be any suitable type or construction. A microactuator 140 is composed of a relatively thin, flexible, biocompatible silicone elastomer, such as polydimethyl siloxane (PDMS).

Referring again to FIGS. 7, 8A and 8B, embedded within the membrane 100 are ferromagnetic pieces 150. The membrane 100 experiences displacement when exposed to an external magnetic field 110 acting on the ferromagnetic pieces 150. For example, in testing it has been shown that the presence of an oscillating 2.85×105-A/m external magnetic field, a 1.2-ml/min flow rate was measured for an actuation frequency of 2.9-Hz. The flow rate is easily varied by adjusting the frequency. Preferably, the constituent components of the diffusers 120 are non-moving and have diverging walls in the positive flow direction (arrows 130) that promotes one-way fluid flow in the positive direction (arrows 130).

For best pump efficiency, diffuser pressure losses in the negative direction (reverse direction of the arrows 130) need to be maximized while minimizing losses in the positive direction (arrows 130). Regarding the magnetic field 110 employed, relatively high magnetic fields (up to 1 Tesla) are used with this design of the present invention pump 30. Although relatively high magnetic fields 110 are employable, it is noted that testing has indicated that magnetic fields as low as 0.11 to 0.23 Tesla, are sufficient in producing relatively large displacements of the membrane 100, and thus produce adequate flow rates in most applications.

Although a microfluidics-type pump 30 including a magnetically-actuated membrane 100 is described above, it is noted that any mechanical (and/or electro-mechanical) microfluidics handling systems having micropumps/nanopumps and/or microvalves/nanovalves that employ various actuation mechanisms are ideal for use with the present invention oral implant 10. Such systems are known in the art. Although the use of unidirectional diffusers 120 located at inlet and outlet areas of the pump 30 to control the direction of flow therethrough, any other suitable device capable of controlling fluid direction can be employed. Alternative actuation principles that can be applied to membrane micropumps include piezoelectric, electrostatic, thermopneumatic, bimetallic and electromagnetic, or combinations thereof. A common material chosen for construction of these devices is silicon, although other suitable materials, such as certain plastics, may be employed, if desired. The types of microvalves and flow controllers that may be used include passive check valves, active diaphragm valves and nozzle diffuser pairs.

Referring again to FIG. 1, since the primary purpose of the oral implant 10 is the delivery of one or more medications, and not the restoration of a lost or diseased tooth, the fabrication of a crown and the placement of the oral implant in a dental arch are not necessary. More specifically, the oral implant 10 is optimally placed in or around the tuberosity area in the maxilla or the retromolar area in the mandible. Both potential sites are outside of the dental arch, reducing the risk for damaging vital structures (e.g., nerves and vessels) during implantation.

Positioning of the oral implant 10 is governed by a relatively small number of restrictions. In general, the oral implant 10 should be positioned such that the implant length is maximized. This is done to maximize the surface area associated with the transmission site 55, as well as facilitate and encourage osseointegration to occur at the desired locations. Additionally, positioning can be governed by the size of the oral implant 10, which, in turn, can depend upon the number, amount and other parameters of the medication(s) to be contained and delivered by the oral implant 10. Finally, positioning of the oral implant 10 can be dictated in part by the need for access to the oral implant 10. Depending upon the maintenance (e.g., number of times it must be accessed, refilled, etc.) associated with the oral implant 10, such factors can influence placement for ease of access (whether by care givers or the individual with the oral implant 10).

The angulation associated with the placement of the oral implant 10 is not of great significance to the functioning of the oral implant 10. Therefore, other considerations, such as aesthetics, and the like, can dictate this parameter of the placement. This feature of the present invention stands in stark contrast to the requirements associated with implants used in traditional dentistry (since angulation and exact location of the implant are not of great importance), making the oral implant 10 a quick, relative easy and relatively inexpensive option for embodying the implantable interface system of the present invention.

Preferably, the upper component 70 takes the overall form of a healing abutment or substantially hollow capsule, but any suitable shape and size, depending upon factors such as the medication(s) to be contained therein and delivered, the cosmetics of the placement, can be utilized. To satisfy the above-identified conditions for placement, the oral implant 10 should be placed at an oblique angle and not in a location where another tooth blocks access to the oral implant 10 for maintenance. Such placement provides significant advantages for the oral implant 10, including, without limitation, placement of the oral implant 10 out of the dental arch, therefore out of occlusion, and not adversely affecting the aesthetics of the arch. Additionally such placement avoids impeding speech or mastication of the individual with the oral implant 10, and does not appear as a foreign object to the tongue.

The materials used for the external surfaces of the oral implant 10 can be any suitable that are used for traditional dental implants, such as various grades of commercially pure titanium or titanium alloys, and the like. The materials used within the oral implant 10 should be biocompatible and non-reactive to the contents of the first chamber 40 and/or the second chamber 60. The internal micro- and/or nano-components can be constructed of any suitable materials employed for such components, such as silicon, polydimethyl siloxane and the like. Finally, materials used to construct the exterior regions of the oral implant 10 that are in direct contact with the tissues of the patient where transmission of material occurs (i.e., the transmission site 55) differ from those used on the remaining exterior portions of the oral implant 10 in that selection of such materials is based upon facilitating transfer of materials at the transmission site 55. Representative examples of such materials include stainless steel, platinum, or other suitable biocompatible material. Use of such materials do not promote osseointegration, and therefore facilitate transmission of materials.

This embodiment of the implantable interface takes the form of the oral implant 10. However, it is noted that the implantable interface of the present invention could take the form of other site-specific implants or the like. The use of an oral implant as an exemplary embodiment does not limit the scope of the present invention to such embodiment. Additionally, it is noted that the foregoing description related to location and positioning of the oral implant 10 applies equally to the various embodiments described herein, and not just to that embodiment shown in FIG. 1.

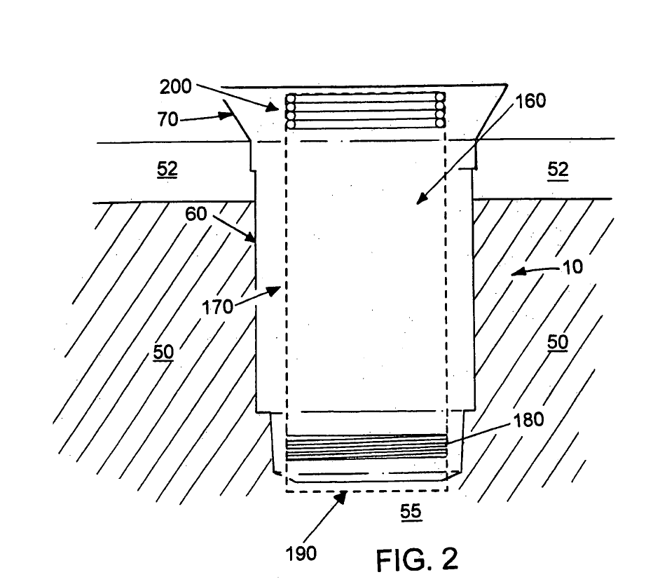

Now referring to FIG. 2, there is shown another embodiment of oral implant 10. In this embodiment, the medication to be delivered via the oral implant 10 is contained within a biodegradable plug 160 and is preferably some type of degradable polymer, or more specifically, a biodegradable polymer, such as a polyvinylalcohol polymer or ethylene vinyl acetate copolymer. Common examples of such a biodegradable plug 160 include ocular scleral plugs.

This embodiment of the oral implant 10 comprises a plug chamber 170 wherein the biodegradable plug 160 rests within the oral implant 10. The biodegradable plug 160 includes a threaded section 180 that screws on the bottom opening 190 of the oral implant 10. A small force spring 200 is incorporated into the upper component 70. The spring 200 functions to push the biodegradable plug 160 downward as it degrades so that the medication is continuously released from the plug chamber 170 at the bottom opening 190 of the oral implant 10 to the transmission site 55 (capillary bed surrounding the apical end of the oral implant 10). After release from the oral implant 10, absorption distributes the released medication to tissue adjacent to the transmission site 55, and, via circulation, it is distributed to the rest of the body. Since the polymer is degradable by the enzymes present in the vicinity of the oral implant 10, gradual release of medication occurs at a controllable rate. It is not uncommon to have ocular plugs maintain a therapeutic dose of a medication for an extended period (e.g., 180 days). However, in the present invention oral implant 10, since there is substantially more room within the interior (plug chamber 170) of the oral implant 10, a steady medication dose program can be maintained for a significantly longer period of time and/or a larger dose for systemic drug delivery may be used.

Another advantage associated with this embodiment of the oral implant 10 is the ease with which maintenance can be performed on the oral implant 10. For example, the replacement of the biodegradable plug 160 is simple and could likely even be accomplished by the user without the need for assistance from a care provider (e.g., health care professional). Both assisted and unassisted maintenance of this embodiment of the oral implant 10 is contemplated herein.

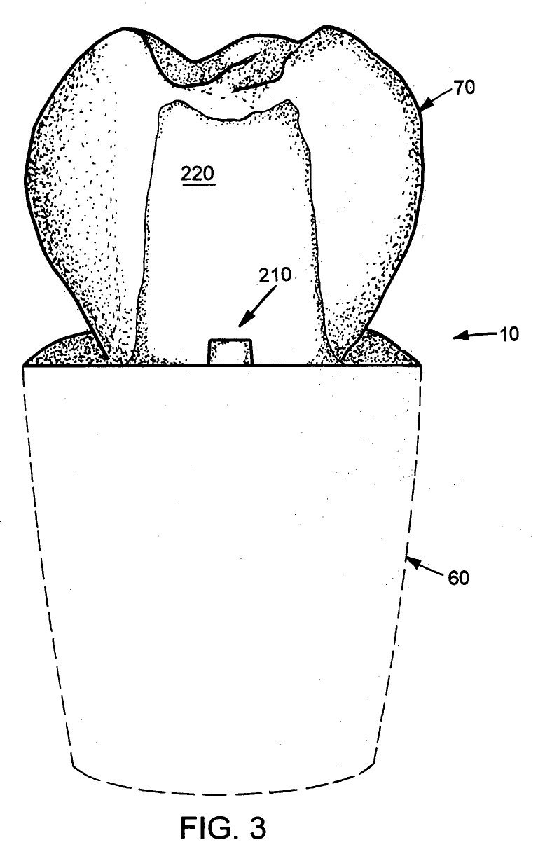

FIG. 3 illustrates an embodiment of the upper component 70 of the oral implant 10 of the present invention. This embodiment of the oral implant 10 includes a microchannel 210 connecting the lower component 60 to an additional chamber 220, providing additional storage for medication to be delivered via the oral implant 10. Using the additional chamber 220 of the upper component 70 with the chamber 40 (FIG. 1) of the lower component 60, this embodiment of the oral implant 10 can deliver two medications to the transmission site 55 associated with the oral implant 10 (FIG. 1). It is noted that although the delivery of two medications is described herein, the present invention implantable interface in the form of the oral implant 10 is capable of delivering any number of combination of medications or other materials via the use of additional chambers, disks, capsules or the like, as desired by the proposed application. The considerably larger amount of space located in the oral implant 10 as compared with other implants (e.g., ocular implants) facilitates this feature of the present invention.

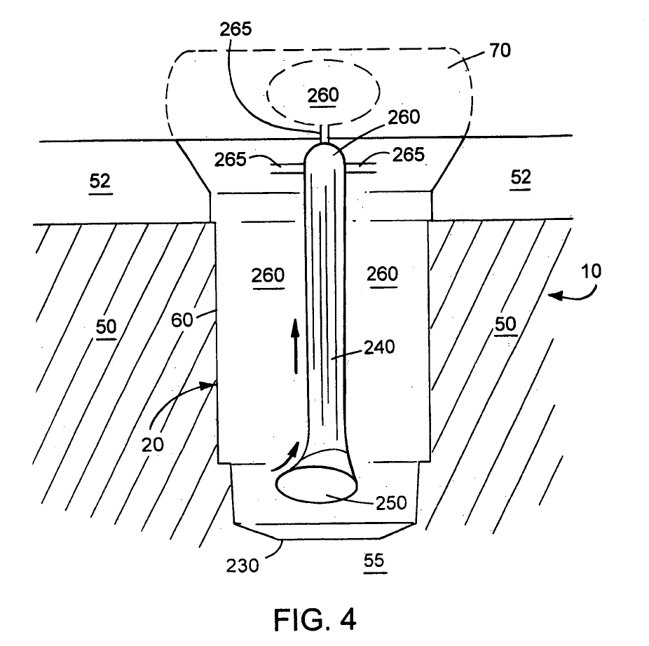

Now referring to FIG. 4, there is shown one embodiment of the oral implant 10 of the present invention configured as a simple blood test system. In this embodiment, the oral implant 10 again includes the main body 20 having two main components: a lower component 60 that serves as an anchor within the maxillary or mandibular bone 50 of a patient (below the gums 52) and stays in place throughout the life of the oral implant 10; and an upper component 70 that is removably attached to the lower component 60, and separates from the lower component 60 so that maintenance (e.g., access to material collected, such as blood) can occur. As with the drug delivery embodiment of the oral implant 10, the upper component 70 is removably attached to the lower component 60 via any suitable means.

A single collection microtube 240 is disposed within the main body 20 of the oral implant 10, and includes a collection end 250. A reservoir 260 is linked to the collection microtube 240 via a microchannels 265. It noted that the reservoir 260 can be located in the lower component 60, the upper component 70 (shown in phantom lines), or both, depending upon the desired application. The collection end 250 of the collection microtube 240 is disposed towards animplanted end 230 of the oral implant 10. Although the relative placement of the collection end 250 with respect to the implanted end 230 is described herein, it is noted that any configuration that would result in the collection of material, such as blood, by the oral implant 10 is suitable for use with the present invention oral implant 10. Additionally, the microtube 240 of this embodiment has a generally tubular, elongated shape and is a single unit. However, any suitable shape and/or number of units that accomplish the collection function can be used, if desired.

In use, the collection end 250 of the microtube 240 receives material, such as blood, from the transmission site 55 (capillary bed surrounding the oral implant 10) and draws it up the microtube 240 to be contained and stored in the reservoir 260. The material being collected is propagated in this embodiment via diffusion over a pressure gradient. Following collection, the upper component 70 can be removed and the material collected accessed for further processing, testing or storage subsequent to collection, depending upon the nature of the material(s) collected. Although collection of a single material, blood in this case, has been described herein, it is noted that the oral implant 10 can be used to collect multiple materials (e.g., tissue, bodily fluids, blood, and its components, like plasma, serum). Additionally, although propagation of material(s) collected is described as occurring via diffusion over a pressure gradient, it is noted that material can be collected via the oral implant 10 via other passive or any suitable active means (e.g., the use of a micropump).

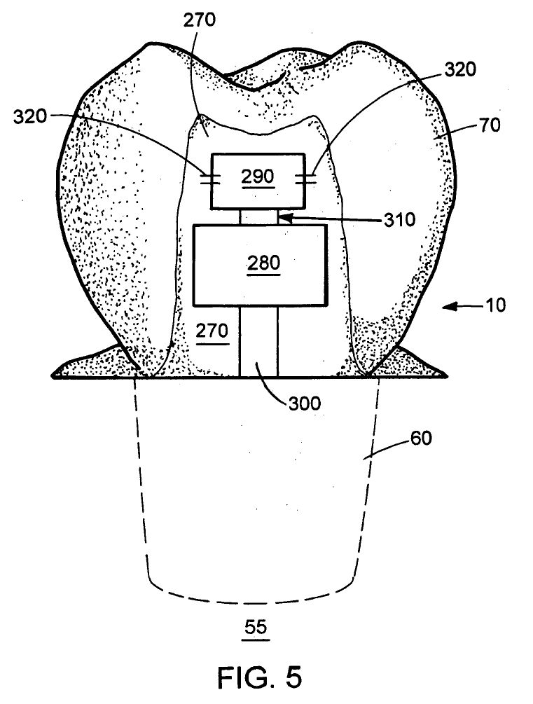

FIG. 5 illustrates a partial cross section of another embodiment of the upper component 70 of the oral implant 10 of the present invention configured as a blood test system including a lab-on-a-chip feature. In this embodiment, the oral implant 10 includes a reservoir chamber 270, a collection pump 280 and a lab-on-a-chip 290. Microchannel 300 connects the collection pump 280 to the transmission site 55 via the lower component 60. Microchannel 310 connects the collection pump 280 to the lab-on-a-chip 290. And microchannels 320 connect the lab-on-a-chip 290 to the reservoir chamber 270.

In use, material, such as blood, is collected from the transmission site 55 and provided from the lower component 60 via microchannel 300 using suction produced by the collection pump 280. The material collected is provided to the lab-on-a-chip 290 so that in situ testing of the material collected can take place. An example of such testing is a diabetic testing blood collected for the sugar levels. Any additional material collected can be provided to the reservoir chamber 270. Such material can be used later to re-run a test, confirm the results obtained from the lab-on-a-chip 290, or the like. Although this embodiment of the oral implant 10 is described using a collection pump to propagate material collected, it is noted that the oral implant 10 is capable of collecting and testing in situ material using any suitable means to propagate collected material.

The lab-on-a-chip 290 is typically a planar device on which a number of chemical processes are performed in order to go from input samples to analyzed results. In such an apparatus, microfluidic channels form complex manifolds for fluid manipulation and controlled delivery of samples of material. Since there is no turbulence in the microfluidic devices, layers of fluids containing different components are able to flow along together and mix rapidly by diffusion only. Planar microfluidics is essentially the fluid equivalent of a printed circuit board in electronics. The usual processes taking place in connection with the lab-on-a-chip 290 are injection, transportation, separation, reactions and detection. Additional microchannels (not shown in FIG. 5) can be used to introduce dilution or chemical reagent(s) into the lab-on-a-chip 290. Depending on the test(s) desired and the desired fate of the resultant fluid(s) there can also be one or more output microchannels that lead to waste chambers or the like. Currently, such systems employ such microfluidics elements as the H-filter and T-sensor, mixers, reactors. The H-filter has two input flows and two outputs and is usually multiplexed to analyze many samples at one time and utilizes diffusion to filter or extract desired components from one of several fluids processed. Diffusion along the horizontal section extracts certain elements out of the sample and into the diluent. The H-filter can separate molecules by size and weight and it extracts small particles from a solution that also contains large particles and separates plasma from blood cells. The T-sensor has multiple input flows and one output flow. The extent of diffusion from one fluid into the other is detectable along the channel. This allows multiple fluid streams to be analyzed simultaneously using diffusion to rapidly differentiate large from small molecules. The T-sensor can therefore perform chemical reactions, provide chemical sensing and molecule detection. Although the term “lab-on-a-chip” has been used herein, it is noted that any self-contained testing apparatus suitable for use in the environment of the oral implant 10 can be used, if desired.

The incorporation of means for wireless communication (uni- or bi-directional) into this embodiment of the oral implant 10 allows the results of any in situ testing to be reported to a remote location, such as a health care professional or the like. Additionally, two-way communication means can be used to remotely control the various features of the oral implant 10, including material collection (e.g., amount, timing), type(s) of testing performed in situ, amounts of reagents or diluents used, etc.

Another embodiment of the oral implant 10 is shown in FIG. 6 in partial cross section, said embodiment including a drug delivery system and a fluid test system. In essence, the embodiment shown in FIG. 6 is one where the embodiments shown in FIGS. 1, 3, 4 and 5 are combined such that the oral implant 10 includes both the material delivery and material collection (and/or in situ testing) features.

In FIG. 6, another embodiment of the oral implant 10 is shown. The material delivery (e.g., drug delivery) feature of this embodiment is located in the lower component 60 and includes a pump 30, a chamber 40, microchannels 330 linking the pump 30 to the surrounding tissue 340 of the mandibular bone 50 (below the gums 52), a microchannel 350 linking the pump 30 with the chamber 40, a microchannel 360 linking the chamber 40 with the reservoir chamber 270, and a microchannel 370 linking the collection pump 280 with the transmission site 55 associated with the oral implant 10. The drug delivery of this embodiment of the oral implant 10 essentially functions as described above in connection with FIGS. 1, 2 and 3.

Now turning to the “lab-on-a-chip” feature of this embodiment located in the upper component 70 of the oral implant 10, the collection pump 280 is linked to the lab-on-a-chip 290 via microchannel 380, and the collection and testing features of this part of the oral implant 10 function as described above in connection with FIG. 5.

The primary advantage to this embodiment of the oral implant 10 is the ability to coordinate and integrate the functions of the material delivery feature and the material collection and testing features. A common example is the use of the oral implant 10 of this embodiment by diabetics. A diabetic could use the oral implant 10 to provide periodic testing of blood sugar utilizing the collection and testing features of the oral implant 10, and use feedback from such testing to control the delivery of insulin using the material delivery features of the embodiment.

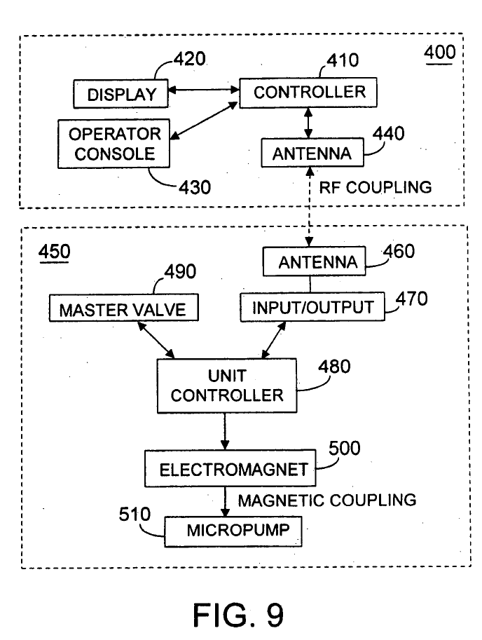

FIG. 9 is a diagram of an electronic control system associated with an embodiment of the oral implant 10 of the present invention. A control system 400 comprises a remote controller 410 connected to a display 420, an operator console 430 and an antenna 440. The remote controller 410, display 420, operator console 430 and antenna 440 can be of any design and type suitable for use with controlling and communication with unit system 450.

The unit system 450 comprises an antenna 460 capable of coupling with the antenna 440 of the control system 400, an input/output device 470, and a unit controller 480. Additional components of the unit system 450 are specific to the embodiment of oral implant 10 being controlled/in communication with the control system 400. In the embodiment shown in FIG. 9, those additional components are a master valve 490, an electromagnet 500 and a micropump 510. Preferably, the antenna 440 of the control system 400 couples with the antenna 460 of the unit system 450 via radio frequency communication. However, any suitable means for coupling the control system 400 to the unit system 450 can be employed, if desired.

In operation, a user utilizes the control system 400, via the operator console 430 (e.g., a terminal, computer or other computing device, such as a personal digital assistant or the like) and the display 420 to send data to and/or receive data from the unit system 450. Examples of data sent include commands to operate the various features and functions of the oral implant 10. Examples of data received from the unit system 450 include test results performed by the oral implant 10.

As a simple example, should a user want to control the unit system 450 of the oral implant 10 to deliver a drug (e.g., hormones) to the body associated with the oral implant 10, the control system 400 would be used to send a signal via antenna 440 to the unit system 450 to cause the master valve 490 to draw a specific amount of drug to be administered, operate the electromagnet 500 to provide a magnetic field that, in turn, causes the micropump 510 to deliver the dosage. Although the above-identified example is an overly-simplified example, the control system 400 operating in concert with the unit system 450 can be used to control all of the features and functions of the oral implant 10, including integration and coordination of same.

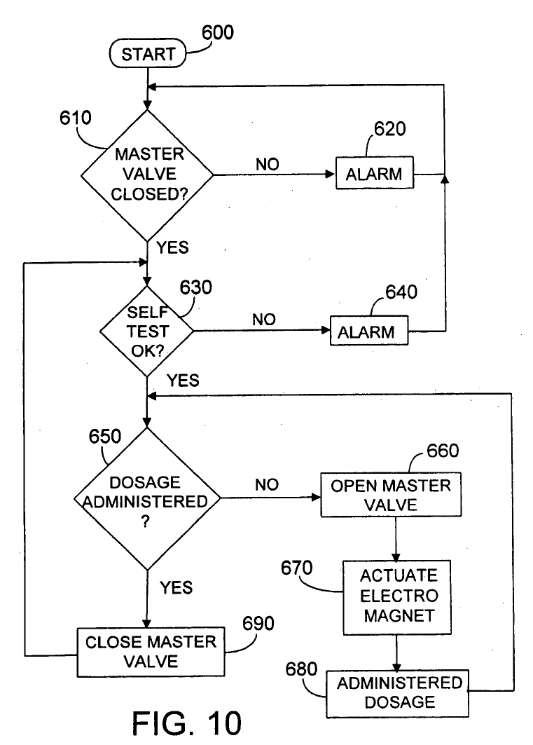

FIG. 10 is a flowchart of the logic flow of a control program associated with the embodiment of oral implant 10 of the present invention illustrated in FIG. 9. Step 600 of the program is a start position. At Step 610, the program determines whether the master valve 490 is closed. If the master valve 490 is open, then an alarm is produced at Step 620 and the program returns to Step 600. If the program determines that the master valve 490 is closed, then it proceeds to Step 630 to perform a self-test. If the self-test performed in Step 630 is not OK, then the program produces an alarm at step 640 and returns to Step 600. If the self-test performed is OK, then the program moves to Step 650 and determines whether a dosage has been administered. If the dosage has not been administered, then the program moves to Step 660 to open the master valve 490, actuate the electromagnet 500 at Step 670 to administer the dosage (Step 680). If at Step 650 it is determined that the dosage has been administered, the program moves to Step 690 to close the master valve 490, and the program returns to Step 630 to perform a self-test.

As illustrated, the program has the ability to self-test and correct the functioning of the oral implant 10, an important feature since the delivery of drugs can be crucial to the health and well-being of a patient. It is noted that the system and program described above are exemplary in nature. A different logic program for each dosage, as well as other functions, would be included.

The inclusion of means for communications in this embodiment provides additional control of and features to the oral implant 10. For example, a diabetic could use the wireless communications feature to control the release of insulin in the event of a sudden change in blood sugar not addressed by the feedback feature or the like. Additionally, the results of blood sugar tests and the amount of insulin delivered by the system could be wirelessly communicated both to the patient, as well as to a remote location, such as a health care provider, a system including artificial intelligence capabilities or the like capable of analyzing the information and acting on same or providing additional information based upon same. Wireless communications means including antenna(s) and other suitable components are known in the art.

Apart from the specific potential implementations identified above, the implantable interface system of the present invention can also be used as an automated immunization/treatment system, the use of which could protect soldiers, even at remote and diverse locations, against chemical or biochemical warfare. Such a system would preferably be of the embodiment type illustrated in FIG. 6 (but could be any of the embodiments outlined herein) and would include a reservoir for containing one or more materials, such as drugs, to be administered under certain, perhaps predetermined conditions to the individual with whom the system was associated. For example, employing the soldier example, as soldiers are deployed over a large geographic area during combat or the like, general command could determine that the troops were subject to an imminent chemical or biological attack. Utilizing any number of communications systems, including, wireless systems, instructions for the delivery of one or more specific antidotes and/or medications contained within the oral implant 10 could be sent out by command and received by the oral implant 10. The result would be the immediate delivery of the appropriate type and level of medication(s)/antidote(s) to far-flung individuals, even those who were unaware (e.g., asleep, unconscious, etc.) of any potential attack. Such a system would allow for appropriate and immediate protection/treatment of such individuals without the need for disruption of those individuals' current responsibilities and/or movement of such individuals or travel for medical personnel to accomplish this result. Although the example of a combat embodiment has been described above, an equally important use of such an embodiment of the present system is in the context of protection from terrorism. Any individual could employ such an embodiment to provide a quick and appropriate administration of antidote(s)/treatment(s) depending upon the terrorism threat faced/experienced by that individual. Parents, guardians and/or caregivers could control (even remotely) such systems for their children, invalid or incapacitated charges.

Another exemplary use of the present invention interface system is as a tracking device for humans (e.g., the elderly, children, Alzheimer patients, etc.), in which miniaturized electronics could be employed to provide transmission of information, potentially coordinated with global tracking systems, such as global positioning systems, about the human associated with the interface. Current tracking devices for use in these contexts suffer from a variety of disadvantages, including relatively large and bulky dimensions, and a lack of tamper resistance.

Yet another exemplary use of the present invention interface system is as a “black-box” capable of monitoring one or more parameters about the human associated therewith, and reporting same, either in real time, or as a recorded history over time. Such a device could be used, for example, to monitor incarcerated individuals or even provide the monitoring necessary to allow qualifying individuals to be under “house arrest” versus actual incarceration. Other uses of such a system, for example to monitor the vital signs of astronauts, are contemplated by the present invention system.

A number of embodiments of the invention have been described. Nevertheless, it will be understood that various modifications may be made without departing from the spirit and scope of the invention. Accordingly, other embodiments are within the scope of the following claims.

What is claimed is:

1. An interface for transmission of materials between an internal environment and an external environment associated with an essentially closed system, said interface comprising:
a main body attachable to the essentially closed system;
means for transmission of materials between the internal environment and the external environment;
said means capable of transmitting materials uni-directionally or bi-directionally between the internal environment and the external environment; and
said means being self-contained within the main body.

2. The interface of claim 1, wherein the essentially closed system is a body.

3. The interface of claim 2, wherein the body is a human body.

4. The interface of claim 2, wherein the body is a non-human body.

5. The interface of claim 1, wherein the main body includes at least one chamber for containing material to be transmitted via the interface.

6. The interface of claim 1, wherein the main body includes at least one chamber for receiving materials transmitted via the interface.

7. The interface of claim 1, wherein the main body is an implant.

8. The interface of claim 7, wherein the implant is an oral implant.

9. The interface of claim 1, wherein the means for transmission is a pump.

10. The interface of claim 9, wherein the pump is a micropump or a nanopump.

11. An interface for transmission of material between an internal environment and an external environment associated with a human body, said interface comprising:
a main body attachable to the human body;
a pump for transmission of material between the internal environment and the external environment;
said pump capable of transmitting material uni-directionally or bi-directionally between the internal environment and the external environment; and
said interface being associated with the human body via implantation.

12. The interface of claim 11, wherein the main body delivers material from the external environment to the internal environment.

13. The interface of claim 11, wherein the main body extracts material from the internal environment and provides it to the external environment.

14. The interface of claim 11, wherein the main body:
extracts material from the internal environment and provides it to the external environment; and
delivers material from the external environment to the internal environment.

15. The interface of claim 13, wherein the material extracted from the internal environment is retained by the main body.

16. The interface of claim 15, wherein the material retained by the main body is subjected to processing within the main body.

17. The interface of claim 16, wherein the processing is selected from the group consisting of: testing, monitoring, sampling and storing.

18. The interface of claim 16, wherein the processing produces results that are retained by the main body.

19. The interface of claim 16, wherein the processing produces results that are communicated to a remote location.

20. The interface of claim 19, wherein the results are communicated to a remote location via means for wireless communications.

21. The interface of claim 14, wherein the main body delivers material from the external environment to the internal environment in response to material extracted by the main body from the internal environment.

22. The interface of claim 11, wherein the material is selected from the group consisting of: drugs, medication, therapeutics, antidotes, fluids and data.

23. The interface of claim 11, wherein the interface further includes means for two-way communications.

24. The interface of claim 23, wherein the means for two-way communications is wireless.

25. The interface of claim 23, wherein the means for two-way communications is capable of controlling the transmission of material between the internal environment and the external environment.

26. The interface of claim 11, wherein the interface is an oral implant.

27. The interface of claim 23, wherein the means for two-way communication is coupled with means for providing information about physical location of the human.

28. The interface of claim 23, wherein the information is provided in real time.

29. The interface of claim 23, wherein the means for providing information is a global positioning unit.

30. The interface of claim 1, wherein the means for transmission of materials is passive.

31. The interface of claim 1, wherein the means for transmission of materials is accomplished via degradation.
